(19) United States
(12) Patent Application Publication
(10) Pub. No.: US 2020/0045962 A1
(43) Pub. Date: Feb. 13, 2020

(54) METHOD FOR PREPARING SURFACTANT COMPOSITIONS COMPRISING ALKYL LIDURONAMIDES D-GLUCURONAMIDES AND L-RHAMNOSIDES FROM ULVANS

(71) Applicant: ÉCOLE NATIONALE SUPÉRIEURE DE CHIMIE, RENNES (FR)

(72) Inventors: Thierry BENVEGNU, RENNES (FR); Nouha SARI-CHMAYSSEM, TRIPOLI (LY); Samir TAHA, BEYROUTH (LB); Hiba MAWLAWI, TRIPOLI (LY)

(21) Appl. No.: 16/468,717

(22) PCT Filed: Dec. 11, 2017
(86) PCT No.: PCT/FR2017/053501

§ 371 Date: Jun. 12, 2019

(30) Foreign Application Priority Data
Dec. 12, 2016 (FR) 1662342

(51) Int. Cl. A01N 43/16; A01N 43/08; C07H 7/033; A01N 25/04

(52) U.S. Cl. CPC A01N 43/16; A01N 25/04; C07H 7/033; A01N 43/08

(57) ABSTRACT

The present invention relates to a novel process for preparing surfactant compositions based on alkyl L-iduronamides, alkyl L-rhamnosides and alkyl D-glucuronamides, to the compositions obtained via said process and to the uses thereof.

### TECHNICAL FIELD

The present disclosure relates to a novel process for preparing compositions comprising alkyl L-iduronamides, alkyl L-rhamnosides and alkyl D-glucuronamides, directly from biobased starting materials (ulvans, green algae) or biocompatible/biodegradable starting materials, to the compositions obtained via said process and to the uses thereof.

The present disclosure finds applications, for example, in the field of surfactants, notably for cosmetics, the plant-protection and agrifood fields, and detergency (industrial).

In the description hereinbelow, the references in square brackets ([ ]) refer to the list of references presented at the end of the text.

### BACKGROUND

In the face of consumer expectations and of ecological and environmental concerns, chemical industrialists have engaged in the development and synthesis of surfactants of plant origin (in other words biobased surfactants), favouring environmentally friendly processes.

In this context, carbohydrate-based surfactants represent an important class of amphiphilic compounds whose growing interest may be explained by functional, economic and environmental factors (Hill and Lehen-Ferrenbach, 2009) [1]. Sugar amide derivatives characterized by the presence of an amide function connecting the hydrophilic sugar head to the lipophilic chain have the advantage of being resistant to hydrolysis in neutral and alkaline media, notably when compared with ester derivatives (Laurent et al., 2011) [2]. Although studies have already shown the possibility of gaining access to amide derivatives from uronic acids such as glucuronic acid and galacturonic acid derived from the hydrolysis of hemicelluloses or pectins (Laurent et al., 2011, mentioned above) [2], there are few studies which make it possible to exploit polysaccharides of algal origin. Only one example of an amide surfactant is derived from the transformation of D-mannuronic acid oligomers originating from the depolymerization of alginates.

Ulvans constitute a family of polysaccharides which have recently been described in green algae of Ulva or Enteromorpha type, these species being present in abundance on the Mediterranean and Brittany coastlines. They are polysaccharides with a unique composition. They are predominantly composed of rhamnose and of uronic acids (L-iduronic acid and D-glucuronic acid), to which elemental units are added glucose and xylose in minor amount. The degree of sulfatation is generally high (5-30%). However, the use of ulvans as sources of L-iduronic and D-glucuronic acids and of L-rhamnose for the potential preparation of monosaccharide surfactants has not been exploited or even envisaged to date.

Three different classes of saccharide-based surfactants exist: esters (sorbitan esters, sucroesters), acetals (alkylpolyglucosides) and amides (alkyl glucamides). Industrially, alkyl sucroamides are produced in two steps: reductive amination of a carbohydrate with an alkylamine, followed by acylation of the resulting N-glycoside (international patent application WO 92/06984; international patent application WO 93/03004; patent EP 0 536 939; U.S. Pat. No. 5,872,111) [3-6]. Similarly, gluconamides are obtained in two steps: oxidation of a carbohydrate leading to a lactone or an aldonic acid, followed by reaction with alkyl amines to form gluconamides (U.S. Pat. No. 2,670,345) [7]. Derivatives including an amide bond between the hydrophilic and lipophilic parts via an N-glycoside bond have more recently been developed (U.S. Pat. No. 7,655,6011) [8]. Another strategy was based on the formation of N-alkylamide surfactants from uronic acids such as glucuronic acid and galacturonic acid derived from the hydrolysis of hemicelluloses or of pectins (Laurent et al., 2011, mentioned above) [2]. All these surfactant synthesis processes use monosaccharides as starting materials and the synthetic conditions are generally environmentally unfriendly (toxic and non-biodegradable reagents). Mannuronamide surfactants have been produced from D-mannuronic acid oligomers (Benvegnu and Sassi, 2010; international patent application WO 03/104248) [9, 10]. The process is based on the production of saturated oligomannuronates (acidic depolymerization) which are then converted into a monosaccharide intermediate including two butyl chains. This synthon is then subjected to an aminolysis reaction using a fatty amine in a solvent such as methanol or isopropanol in the presence or absence of an organic base. The N-acyl surfactant thus obtained has emulsifying properties.

Surfactant compositions comprising alkyl L-guluronamides or a mixture of alkyl L-guluronamides and of alkyl D-mannuronamides have been produced from poly(oligo)guluronates, oligoalginates, alginates and/or brown algae, by following a step of butanolysis and of Fischer glycosylation and a step of aminolysis (Sari-Chmayssem et al., 2016) [13].

There is thus a real need for a novel process for synthesizing compounds and compositions which overcome the defects, drawbacks and obstacles of the prior art, in particular a process for controlling the production at the industrial scale, for reducing the costs and for improving the expected properties of the compounds and compositions, notably in the field of surfactants which can also combine antibacterial and/or antifungal properties, and which satisfy the principle of “blue chemistry”.

### BRIEF SUMMARY

A novel solvent-free process has been developed, using biocompatible/biodegradable reagents, for affording simple access to surfactant compositions based on alkyl L-iduronamide, alkyl L-rhamnoside and alkyl D-glucuronamide, directly from ulvans or from green algae.

Ulvans are extracted, for example, from the green alga Ulva lactuca or Ulva linza by acidic treatment (0.5 M HCl, pH 1.5-2, at 60° C. for 2 hours) followed by precipitation from an alcohol (ethanol), before neutralization with NaOH solution (0.1 M), for example according to the process described by Bay and Lahaye (Carbohydr. Res., 1998, 274, 1-12) [11].

One subject of the present disclosure is thus a process for preparing a composition comprising a mixture of alkyl D-glucuronamides of formula (I) in pyranoside form of formula (Ia) and in furanoside form of formula (Ib), of alkyl L-iduronamide of formula (II) and of alkyl L-rhamnoside of formula (III):

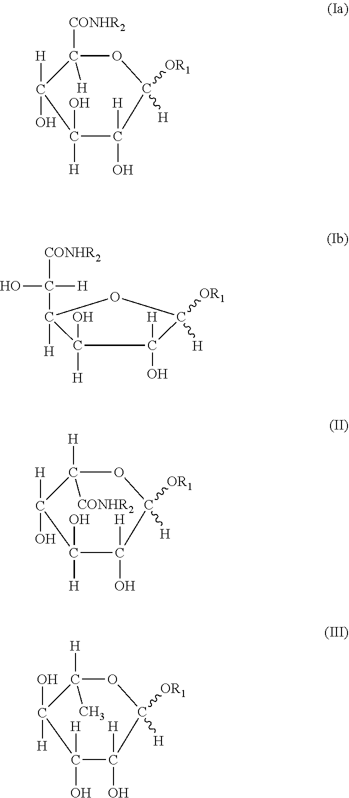

in which
R₁ is a linear or branched, saturated or unsaturated alkyl chain of 2 to 22 carbon atoms;
R₂ is a hydrogen, R₁, a linear or branched, saturated or unsaturated alkyl chain of 2 to 22 carbon atoms including an amine end function,
   and characterized in that said process comprises:
a) a step of butanolysis reaction and of Fischer glycosylation starting with ulvans and/or green algae;
b) a step of aminolysis reaction on the reaction medium obtained from step a), in the presence of a linear or branched, saturated or unsaturated amine of formula R′NH₂ in which R′ is composed of from 2 to 22, preferably from 8 to 18, preferentially from 12 to 18 carbon atoms.

For the purposes of the present disclosure, the term “ulvans” means anionic sulfated water-soluble polysaccharides extracted from green algae of Ulva or Enteromorpha type.

For the purposes of the present disclosure, the term “green algae” means a set of algae whose main photosynthetic pigments are chlorophylls a and b. They regroup various organisms whose sizes may range from a few millimetres to more than a metre and which may be of very varied appearance. Green algae are represented by the following groups: Euglenophyceae, Chlorarachniophyta, Chlorophytes, Chlorokybophyceae, Klebsormidiophyceae, Zygnematophyceae, Chaetosphaeridiophyta, Charophyceae and Coleochaetales. Examples of species of green algae that may be mentioned include: Caulerpa taxifolia, Chara globularis, Ulva lactuca, Ulva linza and Boergesenia forbesii.

According to a particular embodiment of the present disclosure, said process comprises, before step a), a step of preparing the ulvans. The ulvans are derived, for example, from the species Ulva linza and are extracted in their acid form with 0.5 M hydrochloric acid solution (pH=2) heated for 2 hours at 60° C. After centrifugation (removal of the insoluble residues), the supernatant containing the ulvans is purified (removal of the polyphenolic contaminants) by precipitation from ethanol (2.5 to 3 times the volume of the aqueous solution containing the ulvans) and the precipitated ulvans are then neutralized with aqueous 0.1 M NaOH solution and the solution is lyophilized to give the ulvans in the form of sodium salts (white solid). The chemical composition of the ulvan is characterized, for example, by a molar mass of 565 100 g·mol⁻¹, a sulfate content of 17.1% (barium sulfate turbidimetric method) and the following sugar composition (HPLC study after methanolysis in 2M HCl for 4 hours): rhamnose=26.2%; glucuronic acid=11.5%; iduronic acid=3.5%; xylose=5.8% and glucose 1.2%.

According to a particular embodiment of the present disclosure, the step of butanolysis and of Fischer glycosylation a) is performed in the presence (i) of water and/or of an ionic solvent and/or of a eutectic solvent, (ii) of a linear or branched, saturated or unsaturated alcohol ROH containing from 1 to 4 carbon atoms, preferably n-butanol, and (iii) of an acid catalyst, for instance hydrochloric acid, sulfuric acid, an alkyl sulfuric acid such as decyl or lauryl sulfuric acid, a sulfonic acid such as benzenesulfonic acid, para-toluenesulfonic acid, camphorsulfonic acid, an alkylsulfonic acid such as methylsulfonic acid (MSA), decylsulfonic acid, laurylsulfonic acid, sulfosuccinic acid or an alkyl sulfosuccinate such as decyl sulfosuccinate or lauryl sulfosuccinate, perhalohydric acids such as perchloric acid, metals such as iron, oxides thereof or salts thereof, for instance the halides thereof. It is preferably an alkylsulfonic acid or methanesulfonic acid (MSA).

For the purposes of the present disclosure, the term “ionic solvent” means, for example, 1-butyl-3-methylimidazolium chloride [BMI 1-butyl-3-methylimidazolium bromide [BMIM]Br, tris(2-hydroxyethyl)methylammonium methyl sulfate (HEMA) and 1-ethyl-3-methylimidazolium acetate [EMIM]AcO; said ionic solvent typically comprising up to 10% of water.

For the purposes of the present disclosure, the term “eutectic solvent” means systems formed from a eutectic mixture of Lewis or Brönsted acids or bases which may contain a variety of anionic species and/or cationic species. First-generation eutectic solvents were based on mixtures of quaternary ammonium salts with hydrogen bond donors such as amines and carboxylic acids (e.g. quaternary ammonium salt and metal chloride (hydrate).

This step a) is performed, for example, by placing one equivalent of ulvan with a molar mass between 150 000 and 3 600 000 g·mol⁻¹, preferably about 560 000 g·mol⁻¹, derived from Ulva linza or Ulva lactuca; 10 to 1000 molar equivalents of water, and preferably 500 molar equivalents; 2 to 300 molar equivalents of an alcohol as defined above, for example n-butanol, and preferably 150 molar equivalents; 10⁻³ to 10 molar equivalents of an acid catalyst, such as hydrochloric acid, sulfuric acid, an alkyl sulfuric acid such as decyl or lauryl sulfuric acid, a sulfonic acid such as benzenesulfonic acid, para-toluenesulfonic acid, camphorsulfonic acid, an alkylsulfonic acid such as methylsulfonic acid, decylsulfonic acid, laurylsulfonic acid, sulfosuccinic acid or an alkyl sulfosuccinate such as decyl sulfosuccinate or lauryl sulfosuccinate, perhalohydric acids, such as perchloric acid, metals such as iron, oxides thereof or salts thereof, for instance halides thereof, and preferably 1.1 to 10 molar equivalents of alkylsulfonic acid, and preferentially 2.5 molar equivalents of methylsulfonic acid. The reaction is then brought to the reflux point of the azeotrope at atmospheric pressure (Dean-Stark apparatus), between 130 and 135° C. in the case of butanol, preferably over 24 hours. The composition thus formed is predominantly constituted of (n-alkyl)-n-alkyl L-iduronate, (n-alkyl)-n-alkyl D-glucuronate and n-alkyl L-rhamnoside (with, for example, the alkyl group which corresponds to a butyl in the case of the use of butanol).

According to a particular embodiment of the present disclosure, said process may also comprise a step a′) of neutralization of the reaction medium obtained from step a), and performed before step b), leading to a final composition including a variable amount of residual fatty amine salt. For example, the neutralization step is performed in the presence of 1M sodium hydroxide, up to a pH of 7.

The preparation of alkyl L-iduronamides, L-rhamnosides and D-glucuronamides in which the alkyl chain is derived from a fatty amine (for the uronic acids), continues via the aminolysis step b), after lowering the temperature (preferably to 60° C.), adding from 1 to 25 molar equivalents of a linear or branched, saturated or unsaturated amine of formula R′NH₂ in which R′ is composed of from 2 to 22 carbon atoms, and preferably 3 molar equivalents are added. The reaction is performed at a temperature preferably of 65-70° C. and under reduced pressure for the recycling of the alcohol mentioned previously. The aminolysis reaction is performed according to the two protocols below:

1) The Aminolysis Reaction is Performed without Prior Neutralization of the Medium:

In the presence of from 1 to 25 molar equivalents of a linear or branched, saturated or unsaturated amine of formula R′NH₂ in which R′ is composed of from 2 to 22 carbon atoms, and preferably 3 molar equivalents. For example, the fatty amine is chosen from the group consisting of dodecylamine and oleylamine. The reaction is performed at a temperature preferably of 65-70° C. and under reduced pressure for the recycling of the alcohol mentioned previously. The composition thus formed constitutes a working product derived from L-iduronic acid and from D-glucuronic acid in amide form and from rhamnose in glycoside form as emulsifiers. The unreacted salts and sugars may be removed from this composition by taking up in an organic solvent, preferably diethyl ether, followed by filtering and rinsing several times with the organic solvent. The filtrate containing the alkyl L-iduronamides, L-rhamnosides and D-glucuronamides is concentrated to give a composition enriched in products of interest which also constitutes a working product such as an emulsifier having antibacterial and antifungal properties at the concentrations used for the formation of emulsions.

2) The Aminolysis Reaction is Performed after Prior Neutralization of the Medium:

By addition of 1N NaOH solution to a pH close to 7. The medium is then concentrated six-fold under reduced pressure, without concentrating to dryness. From 1 to 10 molar equivalents of a linear or branched, saturated or unsaturated amine of formula R′NH₂ in which R′ is composed of from 2 to 22 carbon atoms, and preferably 1 molar equivalent, are then added. For example, the fatty amine is chosen from the group consisting of dodecylamine and oleylamine. The reaction is performed at a temperature preferably of 65-70° C. and under reduced pressure for the recycling of the alcohol mentioned previously. Thereafter, from 100 to 1000 molar equivalents of water, preferably 500 equivalents, are added to the medium. The mixture is stirred for about 15 minutes at 65-70° C. After stopping the stirring, the medium is left for about 10 minutes at this same temperature so that the organic products flocculate. After lowering the temperature to room temperature, the organic phase solidifies and it is then easy to remove the water charged with the salts via techniques that are well known to those skilled in the art.

According to a particular embodiment of the present disclosure, the preparation of a composition comprising alkyl L-iduronamide, L-rhamnoside and D-glucuronamide derivatives in which the alkyl chain is longer continues via a trans-glycosylation step c) performed on this composition obtained from step b) or on one or more derivatives of this composition isolated/purified via means that are well known to those skilled in the art (e.g. column chromatography on silica gel), for example on the L-rhamnoside derivatives, in the presence of a linear or branched, saturated or unsaturated alcohol of formula R′OH in which R′ is composed of from 2 to 22, preferably from 8 to 18, preferentially from 12 to 18 carbon atoms. For example, the alcohol R′OH is chosen from the group consisting of saturated or unsaturated linear fatty alcohols such as dodecanol and oleyl alcohol. This trans-glycosylation step c) is performed, for example, by introducing into the reaction medium obtained from step b) from 2 to 50 molar equivalents of an alcohol of formula R′OH as defined above, and preferably 15 molar equivalents; from 10⁻³ to 10 molar equivalents of an acid catalyst as defined above, and preferably from 0.1 to 10 molar equivalents of alkylsulfonic acid, and preferentially 1 molar equivalent of methanesulfonic acid. The trans-glycosylation reaction is then continued by allowing the recycling of the short-chain alcohol ROH used previously for the formation of the composition rich in (n-alkyl)-n-alkyl L-iduronate, (n-alkyl)-n-alkyl D-glucuronate and n-alkyl L-rhamnoside. The reaction is performed for 1 hour to 24 hours at a temperature preferably of 70° C. and under reduced pressure for the recycling of the alcohol mentioned previously. The composition thus formed constitutes a working product derived from L-iduronic acid and from D-glucuronic acid in amide form and from rhamnose in glycoside form such as a hydrophone agent, a nonionic detergent or an emulsifier.

According to a particular embodiment of the present disclosure, a step d) of neutralizing the reaction medium obtained from step c), once returned to room temperature and atmospheric pressure, may be performed in the presence (i) of water and (ii) of a base M(OH)x in which M is an alkali metal or alkaline-earth metal, and x is the valency. This step d) is performed, for example, by introducing into the reaction medium obtained from step c), once returned to room temperature and atmospheric pressure, from 0 to 19 molar equivalents of an aqueous solution containing a base of formula M(OH), as defined above, and preferably 2.2 equivalents of 1N sodium hydroxide (NaOH) solution; from 100 to 1000 molar equivalents of water and preferably 780 molar equivalents. Next, the whole is heated at 80° C. with vigorous stirring for 15 minutes. Once the mixture has returned to room temperature, the aqueous phase is separated from the organic phase. Said organic phase is then dried by azeotropic distillation of the water using butanol. The excess alcohol of formula R′OH present in the crude organic product may be partially or totally removed by molecular distillation. After optional purification by chromatography on silica gel (97/3 and then 96/4 and then 90/10 CH₂Cl₂/MeOH), a mixture of products is obtained. By way of example, the mass composition is approximately: 10% alkyl L-iduronamides, 50% alkyl L-rhamnosides and 40% alkyl D-glucuronamides.

The compositions thus formed via the process of the disclosure constitute working products derived from L-iduronic acid and from D-glucuronic acid in amide form and from rhamnose in glycoside form, such as emulsifiers with antibacterial and/or antifungal properties at the concentrations used for the formation of an emulsion.

A subject of the present disclosure is also a composition obtained via a process according to the disclosure. The compositions of the disclosure consist of L-iduronic acid and D-glucuronic acid derivatives in amide form and of rhamnose in glycoside form. In addition, the D-glucuronic acid amide derivatives are in the form of both pyranosides (six-membered rings) and furanosides (five-membered rings), whereas the L-iduronic acid amide derivatives and L-rhamnose glycosides are exclusively in the form of pyranosides. Depending on the chain length and on the nature of the alkyl chains, the compositions of the disclosure are considered as emulsifiers for water-in-oil (W/O) or oil-in-water (O/W) emulsions. Furthermore, they may have antibacterial and antifungal properties.

A subject of the present disclosure is also the use of a composition according to the disclosure as a surfactant. Preferably, said surfactant is chosen from the group consisting of solubilizers, hydrotropes, wetting agents, foaming agents, emulsion-forming agents, emulsifiers and/or detergents.

A subject of the present disclosure is also the use of a composition according to the disclosure as an antibacterial and/or antifungal agent.

A subject of the present disclosure is also a surfactant comprising a composition according to the disclosure. Said surfactant may have the following properties:

| Number of carbon | |
|---|---|
| atoms in the lipophilic | |
| chain (alkyl R₂): | Surfactant |
| Between 1 and 6 | Hydrotropic and/or solubilizing agents |
| Between 6 and 14 | Oil-in-water (O/W) and/or water-in-oil (W/O) |
| | emulsifiers |
| Between 16 and 22 | Water-in-oil (W/O) emulsifiers |

A subject of the present disclosure is also an antifungal and/or antibacterial agent comprising a composition according to the disclosure.

The process of the disclosure leads to novel surfactant compositions using exclusively biobased starting materials (ulvans, green algae) or biocompatible/biodegradable starting materials:
by performing a methodology which allows the transformation of L-iduronic acid and D-glucuronic acid, i.e. the two constituent uronic sugars of ulvans, in addition to rhamnose, to give amide surfactant compositions consisting exclusively of L-iduronic acid and D-glucuronic acid derivatives in amide form and of L-rhamnose in glycoside form;
by proposing conditions which satisfy the principles of blue chemistry, reactions free of organic solvents other than the reagent alcohols/amines, which produce no waste (recycling of the short-chain alcohols (n-butanol, etc.)) and which use biodegradable reagents (methanesulfonic acid and the like);
by performing all of the reactions via a “one-pot” process without isolation or purification of the reaction intermediates, to afford access to the surfactant compositions of the disclosure directly from the ulvans;
by using simple conditions for the partial purification of the crude reaction products and for isolation of the surfactant compositions which make it possible to obtain the derived compounds and the compositions at prices that are more competitive than the current market.

The process of the disclosure thus makes it possible to produce compositions derived from L-iduronic acid and D-glucuronic acid in amide form and from rhamnose in glycoside form which have the advantage of forming water-in-oil (W/O) and oil-in-water (O/W) emulsions that are very stable in comparison with commercial emulsifiers, and of having antibacterial and antifungal properties at the concentrations used for the formation of the emulsions.

Thus, the process of the disclosure makes it possible both to reduce the production costs of surfactant compositions and to propose novel compositions for the purpose of improving the performance qualities (notably emulsifying properties). The presence of the uronic sugars and of rhamnose contributes towards providing advantageous biological activities in addition to the surfactant properties. In particular, many receptors specific for rhamnose exist in human cells and in particular skin cells, namely keratinocytes, and endothelial cells which regulate the inflammatory response. The presence of rhamnoside in the surfactant composition may thus provide biological activities that can be exploited in several fields and notably in cosmetics.

Other advantages may also appear to a person skilled in the art on reading the examples below, illustrated by the attached figures, which are given as illustrations.

### BRIEF DESCRIPTION OF THE FIGURES

FIG. 1 represents the measurement of the emulsifying power of the surfactant composition UlvC₄N₁₂ (A) W/O emulsion and (B) O/W emulsion, in comparison with commercial references Montanov® and Xyliance®.

### DETAILED DESCRIPTION AND EXAMPLES

### Example 1: Process for Obtaining Compositions Based on Alkyl L-Iduronamides, Alkyl D-Glucuronamides and Alkyl L-Rhamnosides from Ulvans

Preparation of the Starting Materials:

The process for extracting the ulvans involves treating green algae with 0.5 M hydrochloric acid solution (pH=2) heated for 2 hours at 60° C. After centrifugation (removal of the insoluble residues), the supernatant containing the ulvans is purified (removal of the polyphenolic contaminants) by precipitation using ethanol (2.5 to 3 times the volume of the aqueous solution containing the ulvans) and the precipitated ulvans are then neutralized with aqueous 0.1 M NaOH solution and the solution is lyophilized to give the ulvans in the form of sodium salts (white solid). By way of example, the chemical composition of the ulvan extracted from the species Ulva linza is characterized by a molar mass of 565 100 g·mol⁻¹, a sulfate content of 17.1% (barium sulfate turbidimetric method and the following sugar composition (HPLC study after methanolysis in 2M HCl for 4 hours): rhamnose=26.2%; glucuronic acid=11.5%; iduronic acid=3.5%; xylose=5.8% and glucose=1.2%.

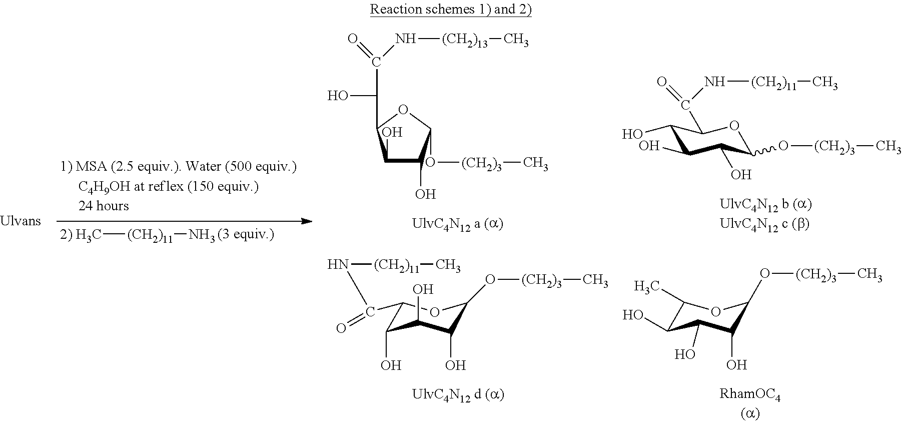

1) Butanolysis and Fischer Glycosylation Reaction

2 g of sodium ulvan extracted from Ulva linza, molecular mass=565 100 g/mol (11.4 mmol, 1 eq) were mixed with 3 mL of distilled water and 1.85 mL of methanesulfonic acid (28.51 mmol, 2.5 eq). 156 mL (150 eq) of butanol were added to the ulvan solution with stirring. The medium was stirred at the reflux temperature of butanol (130-135° C.) for 24 hours. The waters added for the dissolution of the polysaccharide and those formed during the reaction were removed in Dean-Stark apparatus filled with butanol, via water-butanol azeotropic distillation. Since water is denser than butanol, it moves to the bottom of the Dean-Stark apparatus and a few ml of butanol pass into the flask to conserve the initial volume. After 24 hours, thin-layer chromatography (95/5 v/v CH₂Cl₂/CH₃OH) and proton and carbon NMR were performed for the reaction medium to ensure that the expected product had indeed been synthesized.

2) Aminolysis Reaction (without Prior Neutralization of the Reaction Medium Before the Aminolysis Reaction)

The temperature of the medium was lowered to 60° C., followed by addition of 3 molar equivalents of dodecylamine C₁₂H₂₅NH₂ (34.21 mmol, 7.86 g) required to increase the pH to 8.5. After stirring for 30 minutes at 65° C. under a reduced pressure of 150 mbar, the butanol was evaporated off by reducing the pressure from 150 mbar to 6 mbar over a period of 1 hour. The medium was left under a reduced pressure of 6 mbar and at 65° C. for 1 hour 30 minutes to ensure the evaporation of the traces of butanol that were formed.

The residue obtained was taken up in diethyl ether and then filtered through a sinter and rinsed several times with diethyl ether to remove the salts and the unreacted starting sugar. The filtrate (containing butyl rhamnoside and dodecyl glucuronamide and iduronamide) is concentrated under vacuum to give a dark brown oil.

After optional chromatography of the oil obtained on a column of silica gel (80 g, using 95/5 v/v CH₂Cl₂/CH₃OH as eluent), the presence of 0.76 g (3.45 mmol, 31%, C₁₀H₂₀O₅, 220.27 g/mol) of n-butyl α-L-rhamnopyranoside (RhamOC₄) was identified, along with the presence of 0.73 g (1.75 mmol, 16% yield) of a mixture of four isomeric forms of chemical formula C₂₂H₄₃NO₆ and of molar mass=417.59 g/mol, monosaccharide surfactant composition named UlvC₄N₁₂. In the case of the D-glucuronic acid surfactant derivatives present in ulvan, 1D and 2D NMR experiments showed the presence of two isomers, a (H-1: 4.92 ppm, J_{1.2}=3.8 Hz, C-1: 98.62 ppm) and β (H-1: 4.37 ppm, J_{1.2}=7.8 Hz, C-1: 102.89 ppm) in pyranose form, and an α furanose form (H-1: δ 4.98 ppm, C-1: 108.67 ppm). The L-iduronic acid present in ulvan appears to lead to an α pyranose form (H-1: 4.95, J_{1.2}=0.9 Hz, C-1: 108.15).

After having determined the chemical shift of the H-1 anomeric proton of each of the isomers and of the anomers thereof, the proportion of each of the four forms present in the UlvC₄N₁₂ mixture was evaluated from the ¹H NMR spectrum by integration of the signals relating to the anomeric proton of each of the four forms obtained. The surfactant composition UlvC₄N₁₂ is then formed from n-(12-dodecyl)-n-butyl α-D-glucurofuranosiduronamide (47%), n-(12-dodecyl)-n-butyl α-D-glucuropyranosiduronamide (26%), n-(12-dodecyl)-n-butyl β-D-glucuropyranosiduronamide (7%), n-(12-dodecyl)-n-butyl α-L-iduronopyranosiduronamide (20%). The proportions of the furanose form (α) and of the pyranose forms (α and β) in the UlvC₄N₁₂ mixtures made it possible to evaluate a pyranose/furanose ratio. The value of the pyranose/furanose ratio is of the order of 1.12 for the UlvC₄N₁₂ mixture indicating that the pyranose forms (α and β) of n-dodecyl n-butyl D-glucuronamide and n-dodecyl n-butyl L-iduronamide are predominant relative to the α furanose form of n-dodecyl n-butyl D-glucuronamide.

On account of the different polarities, it was possible to separate by column chromatography on silica gel (eluent: 95/5 dichloromethane/methanol) the uronamide compositions (UlvC₄N₁₂) from the more polar n-butyl L-rhamnoside compound (RhamOC₄).

The molar mass of the n-butyl α-L-rhamnopyranoside compound (220.27 g/mol evaluated by mass spectrometry) and the absence of an absorption band characteristic of sulfate functions in its infrared spectrum (at 1260 cm⁻¹) showed that the sulfate group initially present on the rhamnose unit of ulvan is released under the effect of the acidic conditions (pH=1.5) during the first step of the process (butanolysis and/or hydrolysis, glycosylation, esterification).

3) Trans-Glycosylation Reaction Starting with Butyl L-Rhamnoside Isolated During the Separation by Column Chromatography on Silica Gel

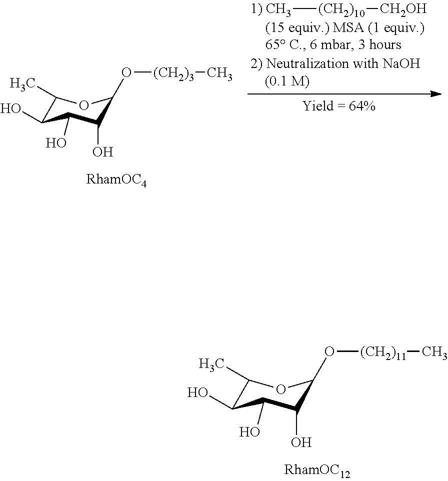

The n-butyl α-L-rhamnopyranoside (0.5 g, 2.27 mmol, 1 eq.), separated from the surfactant composition UlvC₄N₁₂ by column chromatography on silica gel, was taken up in dodecanol (15 eq.) in the presence of one equivalent of MSA (2.27 mmol, 148 μL). The trans-glycosylation was then performed for 3 hours at 65° C. under reduced pressure (6 mbar) in sufficiently dilute medium so as to avoid the degradation of the butyl rhamnoside. At the end of the reaction, the reaction medium was allowed to cool and was then neutralized with an NaOH solution (0.1 M).

The difference in polarity between the compound n-dodecyl α-L-rhamnopyranoside, having a hydrophobic chain containing 12 carbon atoms, and n-butyl α-L-rhamnopyranoside made it possible to separate these two compounds by column chromatography on silica gel, using a 95/5 dichloromethane/ethanol mixture as eluent. The trans-glycosylation yield (=64%, 0.48 g) was evaluated from the molar masses of n-butyl α-L-rhamnopyranoside (220.27 g·mol⁻¹) and n-dodecyl α-L-rhamnopyranoside (C₁₈H₃₆O₅, 332.48 g·mol⁻¹).

The 1D and 2D NMR results confirm the structure of n-dodecyl L-rhamnoside. The proton NMR spectrum showed the presence of a dodecyl chain in the anomeric position (doublet of triplets at 3.38 and 3.65 ppm corresponding to the protons of the 0-CH₂ function bonded in the anomeric position on rhamnose (O—CH₂: 67.89 ppm). The doublet at δ 4.75 ppm for the anomeric proton H-1 corresponds to the α anomer of dodecyl L-rhamnoside 2=2.1 Hz). The anomeric carbon C-1 of this compound RhamOC₁₂ gives a signal at 99.77 ppm. Furthermore, the HMBC 2D NMR spectrum showed a correlation between the anomeric proton H-1 (4.75 ppm) and the carbon ones of the O—CH₂ function bonded in the anomeric position on rhamnose (67.89 ppm).

### Example 2: Measurement of the Interface Tensions of the Surfactant Compositions Based on Alkyl L-Iduronamides and Alkyl D-Glucuronamides from Ulvans

The interface properties of the surfactant composition UlvC₄N₁₂ were evaluated by measuring the oil-water interface tensions. The surfactants were dissolved in sunflower oil at concentrations ranging from 0.12 to 0.46 g/L. In order to promote the solubility of the surfactants in the oil, the solutions were left in an ultrasonic bath for 10 minutes at 50° C. The interface tension measurements were taken between Milli-Q water and the solutions of sample in oil.

The tensions at the interface between the oil and the water were measured at 25° C. with a ring tensiometer (Krüss, K 100C model). The ring used was horizontally-suspended calibrated iridium-treated platinum. Before each measurement, the ring was cleaned meticulously and flame-dried. The sample goblet is a cylindrical glass container placed in a heat-regulated chamber.

The interface tension between the sunflower oil (Carrefour brand) and water at 25° C. ranged between 24.71 and 25.04 mN/m.

For each concentration of the surfactant composition, the machine initially measured the surface tension of sunflower oil containing the surfactant (low-density liquid) and then the surface tension of water (high-density liquid). Finally, the oil was added delicately to the water, while avoiding the formation of bubbles, and the machine began measuring the interface tension between the sunflower oil and the water (average of 10 measurements).

Interface tensions of the surfactant composition UlvC₄N₁₂ (mN/m)]

| | UlvC₄N₁₂ |
|---|---|
| 0.12 g · L⁻¹ | 17.45 |
| 0.25 g · L⁻¹ | 13.89 |
| 0.46 g · L⁻¹ | 10.32 |

The surfactant composition UlvC₄N₁₂ is capable of reducing the interface tension to a value of 10.32 mN/m for a concentration of 0.46 g/L to give the composition emulsifying power.

### Example 3: Measurement of the Emulsifying Power of Surfactant Compositions Based on Alkyl L-Iduronamides and Alkyl D-Glucuronamides from Ulvans

The stability of the oil-in-water (O/W) and water-in-oil (W/O) emulsions formed from the surfactant composition UlvC₄N₁₂ was studied in comparison with that of commercial alkylpolyglycosides: Montanov 82® from SEPPIC and Xyliance® from Soliance/ARD.

The stability of the two types of emulsion, O/W and W/O, was evaluated considering the two water/oil ratios 75/25 and 25/75, respectively, in round-bottomed graduated tubes, 0.5% of the surfactant product is introduced (20 mg). The sunflower oil was introduced (1 or 3 mL) and the surfactants were then dissolved in an ultrasonic bath for 10 minutes at 50° C. After dissolution of the emulsifier, ultrapure water was added (1 or 3 mL).

The two phases were then emulsified using an Ultra-Turrax IKA T18 Basic® homogenizer for 10 minutes at 11 000 rpm. The emulsion was placed in a bath thermostatically regulated at 20° C.

The evolution of the emulsion and its gradual demixing was observed for a few hours to several weeks.

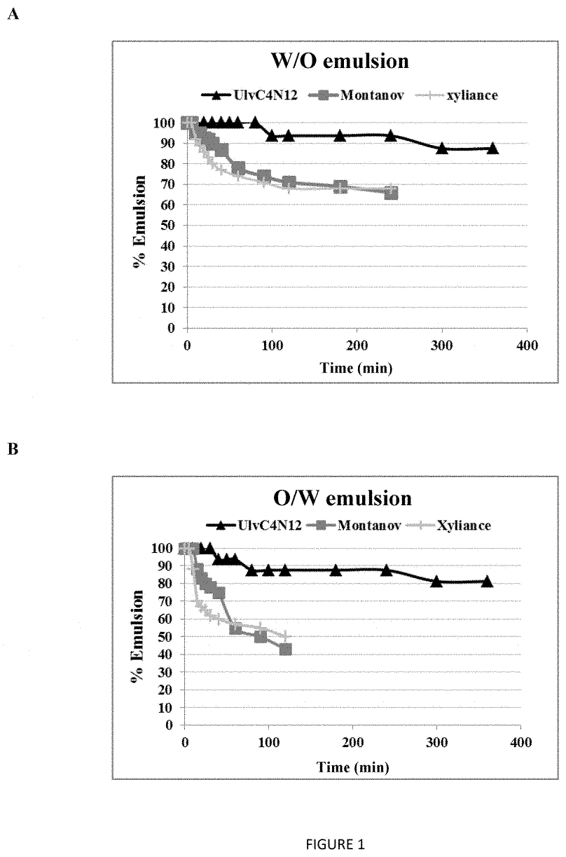

FIG. 1 shows the results of analysis of the emulsifying power of the compositions of the disclosure.

The surfactant composition UlvC₄N₁₂ derived from dodecylamine gave an O/W emulsion characterized by high stability ranging from several weeks to several months. Furthermore, the W/O emulsion formed by the product UlvC₄N₁₂ is very stable.

These experiments made it possible to demonstrate the good stability of the emulsions formed by the monosaccharide surfactant composition UlvC₄N₁₂. Specifically, this surfactant composition has better emulsifying properties than those of Montanov® and Xyliance®, since it makes it possible to form emulsions (W/O and O/W) that are very stable ranging from several weeks to several months, which is not the case for those obtained with the commercial references.

| Type of emulsion | W/O emulsion | O/W emulsion |
|---|---|---|
| UlvC₄N₁₂* | +++ | +++ |
| Montanov ®* | + | − |
| Xyliance ®* | − | − |

Evaluation of the stability of the emulsion, from a few hours to a few months, in demixing as a function of the type of the emulsion.
   *Time for total of the emulsion: −<12 hours; +<24 hours; ++=7 days; +++>1 month.

### Example 4: Antibacterial Activity of Surfactant Compositions Based on Alkyl L-Iduronamides, Alkyl D-Glucuronamides and Alkyl L-Rhamnosides from Ulvans

Two protocols were used. The first (protocol A) was applied to n-butyl L-rhamnoside (RhamOC₄) isolated by chromatography on silica gel. The second (protocol B) was followed to test the activity of the surfactant composition UlvC₄N₁₂ derived from dodecylamine.

### Protocol A: Method of Diffusion on Agar in Petri Dishes

1) Preparation of the Culture Medium:

The culture medium used was a mixture of 21 g/L of Muller-Hinton broth and 10 g/L of agar in water. This mixture was stirred and then left to boil. Next, a step of autoclaving of this mixture for 30 minutes was necessary in order to sterilize it before any manipulation. This culture medium was poured, while hot, into Petri dishes and then left to cool.

2) Preparation of the Test RhamOC₄:

5 milligrams of RhamOC₄ were dissolved in 1 mL of DMSO. Twofold serial dilution with DMSO was then performed starting with the stock solution, so as to obtain the concentrations 2.5 g·L⁻¹, 1.25 g·L⁻¹, 0.625 g·L⁻¹ and 0.3125 g·L⁻¹.

3) Preparation of the Bacterial and Fungal Suspensions:

The bacterial strains used were Pseudomonas aeruginosa, Escherichia coli, Enterococcus faecium and Staphylococcus aureus, in addition to the fungal strain Candida albicans. 10⁶ bacteria were collected and then transferred into a 0.9% NaCl solution. Each Petri dish, containing the Muller-Hinton medium, was inoculated with a different bacterial suspension.

4) Protocol:

After having left the bacterial suspensions to dry on the agar, 10 μL of the test solution (RhamOC₄), at various concentrations, were deposited on the surface of the agar inoculated with the bacterial suspension. 10 μL of DMSO were placed in each Petri dish as negative control.

The positive controls used were discs soaked with ampicillin for Escherichia coli and Enterococcus faecium, ceftazidim discs for Pseudomonas aeruginosa and vancomycin discs for Staphylococcus aureus.

After drying, the Petri dishes were finally incubated at 37° C. in an oven for 24 hours. The antibacterial activity was evaluated by measuring the clarification zone in mm around the place of deposition of the various concentrations of the test RhamOC₄ solution.

### Results:

| | Concentration | P. | | | C. | |
|---|---|---|---|---|---|---|
| | (mg · mL⁻¹) | aeruginosa | E. coli | E. faecium | albicans | S. aureus |
| RhamOC₄ | 5 | 4 | 9 | 6 | 18 | 10.5 |
| | 2.5 | 0 | 4 | 4 | 14 | 5 |
| | 1.25 | 0 | 0 | 0 | 5 | 5 |
| | 0.625 | 0 | 0 | 0 | 0 | 0 |
| | 0.3125 | 0 | 0 | 0 | 0 | 0 |
| Positive | — | Ceftazidim | Ampicillin | Ampicillin | — | Vancomycin |
| control | | (28 mm) | (22 mm) | (26 mm) | | (19 mm) |

The rhamnoside RhamOC₄ showed a very good capacity to inhibit the growth of the gram-positive bacterium Staphylococcus aureus and the yeast Candida albicans. Its power against Enterococcus faecium (6 mm at 5 mg·mL⁻¹) was mediocre. Furthermore, the rhamnoside RhamOC₄ showed inhibitory activity on the gram-negative bacterium Escherichia coli at concentrations of 2.5 and 5 mg·mL⁻¹ with poor inhibitory power on the growth of Pseudomonas aeruginosa.

### Protocol B: Method for Evaluating the Number of Live Bacteria

The antibacterial and antifungal activities of the surfactant composition UlvC₄N₁₂ were evaluated. In this context, the capacity of this monosaccharide surfactant composition to kill bacteria was studied by counting the number of live bacteria on Muller-Hinton agar.

1) Preparation of the Bacterial and Fungal Inoculum

The inoculum was prepared at a turbidity equivalent to 0.5 MacFarland (Biomérieux France), and then diluted to 1/100 (10⁶ CFU/ml)

From this inoculum, a series of dilutions 10⁻¹, 10⁻², 10⁻³, 10⁻⁴, 10⁻⁵, 10⁻⁶ was produced.

100 μl of each dilution were plated (counting method) onto the surface of a Muller-Hinton agar (determination of the number of bacteria as CFU/ml in the inoculum ‘N’).

2) Preparation of the Test Surfactants

A stock solution was prepared for the surfactant composition UlvC₄N₁₂ (203 mg·mL⁻¹). A series of twofold dilutions with DMSO was prepared in Muller-Hinton broth, the final dilution being 1/128.

3) Protocol

1 ml of bacterial inoculum was added to each tube of the surfactant dilutions. After incubation for 24 hours at 36° C. 100 μl of each clear tube were plated onto the surface of a Muller-Hinton agar followed by incubation for 24 hours at 37° C.

The number of live bacteria: N0=n×10 CFU/ml (n=number of colonies) was determined.

The percentage of live bacteria was calculated: N0/N×100.

### Results:

The minimum concentration for 100% inhibition of Enterococcus faecium and Candida albicans was of the order of 1.58 mg·mL⁻¹ for the monosaccharide surfactant composition based on D-glucuronic acid and L-iduronic acid.

As regards the two gram-negative bacteria Pseudomonas aeruginosa and Escherichia coli, very high concentrations of UlvC₄N₁₂ were required to inhibit these two bacteria to 100%, showing that UlvC₄N₁₂ (25.375 mg·mL⁻¹) has poor antibacterial power against these two types of bacteria.

1—These studies of the antibacterial power (Protocols A and B) clearly showed that the gram-positive bacteria Enterococcus faecium and Staphylococcus aureus and also the yeast Candida albicans were more sensitive to the rhamnoside RhamOC₄ and to the amide surfactant composition UlvC₄N₁₂ than the gram-negative bacteria. Specifically, the gram-positive bacteria are characterized by the presence of a very thick layer of peptidoglycan in their cell membrane, in contrast with that of the gram-negative bacteria. The hydrogen bonds between the cell wall of the gram-positive bacteria and the hydrophilic part of the surfactants are then stronger than in the case of the gram-negative bacteria. The hydrophobic carbon chain, the hydrophilic heads of which are anchored in the thick peptidoglycan membrane, could thus interact with the lipid membrane of the gram-positive bacterium, thus promoting its deformation and thereafter the bacterial cell death (Reis et al., J. Brazilian Chem. Soc., 19 (6), 1065-1072,2008) [12].

| Name of the | Name of the | |
|---|---|---|
| surfactant | bacterium | Results |
| UlvC₄N₁₂ 19 | P. aeruginosa | 101.5 mg · mL⁻¹ => inhibition of 100% |
| | | of the bacteria |
| 203 mg/mL | | 50.75 mg · mL⁻¹ => inhibition of 100% |
| | | of the bacteria |
| | | 25.375 mg · mL⁻¹ => inhibition of 99.9% |
| | | of the bacteria |
| | E. coli | 101.5 mg · mL⁻¹ => inhibition of 100% |
| | | of the bacteria |
| | | 50.75 mg · mL⁻¹ => inhibition of 100% |
| | | of the bacteria |
| | | 25.375 mg · mL⁻¹ => inhibition of 99.99% |
| | | of the bacteria |
| | E. faecium | 1.58 mg · mL⁻¹ => inhibition of 100% |
| | | of the bacteria |
| | C. albicans | 1.58 mg · mL⁻¹ => inhibition of 100% |
| | | of the bacteria |

1) Process for preparing a composition comprising a mixture of alkyl D-glucuronamides (I) in pyranoside form of formula (Ia) and in furanoside form of formula (Ib), of alkyl L-iduronamide of formula (II) and of alkyl L-rhamnoside of formula (III): 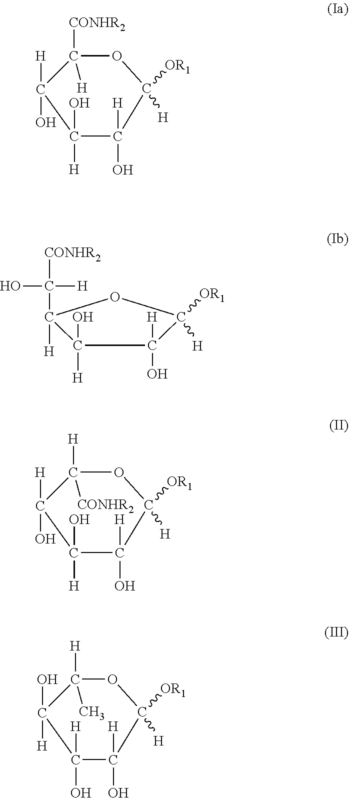
in which
R₁ is a linear or branched, saturated or unsaturated alkyl chain of 2 to 22 carbon atoms;
R₂ is a hydrogen, R₁, a linear or branched, saturated or unsaturated alkyl chain of 2 to 22 carbon atoms including an amine end function,
and characterized in that said process comprises:
a) a step of butanolysis reaction and of Fischer glycosylation starting with ulvans and/or green algae;
b) a step of aminolysis reaction on the reaction medium obtained from step a), in the presence of a linear or branched, saturated or unsaturated amine of formula R′NH₂ in which R′ is composed of from 2 to 22, preferably from 8 to 18, preferentially from 12 to 18 carbon atoms.

2) Process according to claim 1, in which said process comprises a step a′) of neutralizing the reaction medium obtained from step a) before step b).

3) Process according to claim 1, in which step a) is performed in the presence (i) of water and/or of an ionic solvent and/or of a eutectic solvent, (ii) of a linear or branched, saturated or unsaturated alcohol of formula ROH, containing from 1 to 4 carbon atoms, and (iii) of an acid catalyst.

4) Process according to claim 3, in which the acid catalyst is chosen from the group consisting of: hydrochloric acid, sulfuric acid, an alkyl sulfuric acid, a sulfonic acid, an alkylsulfonic acid or an alkyl sulfosuccinate, perhalohydric acids, metals, oxides thereof or salts thereof such as the halides thereof.

5) Process according to claim 4, in which the acid catalyst is methanesulfonic acid.

6) Process according to claim 1, in which the alcohol ROH is n-butanol.

7) Process according to claim 1, in which step b) is performed in the presence of a fatty amine chosen from the group consisting of dodecylamine and oleylamine.

8) Process according to claim 1, said process also comprising:
c) a step of trans-glycosylation of the reaction medium obtained from step b) or of at least one of the isolated derivatives thereof, with a linear or branched, saturated or unsaturated alcohol of formula R′ OH containing from 5 to 22 carbon atoms; and
d) optionally a step of neutralizing the reaction medium obtained from step c) in the presence of water and of a base M(OH)x in which M is an alkali metal or alkaline-earth metal, and x is the valency.

9) Process according to claim 8, in which the alcohol R′ OH is chosen from the group consisting of dodecanol and oleyl alcohol.

10) Process according to claim 8, in which the trans-glycosylation step c) is performed at 70° C. under reduced pressure so as to recycle the alcohol ROH.

11) Composition obtained via a process according to claim 1.

12) Composition according to claim 11, in which said composition is an oil-in-water or water-in-oil emulsion.

13) Composition according to claim 11 configured as a surfactant.

14) Composition according to claim 13, in which the surfactant is chosen from the group consisting of solubilizers, hydrotropes, wetting agents, foaming agents, emulsion-forming agents, emulsifiers and/or detergents.

15) Composition according to claim 11, for use as an antibacterial and/or antifungal agent.

16) Surfactant comprising a composition according to claim 11.

17) Antibacterial and/or antifungal agent comprising a composition according to claim 11.
